(19) United States
(12) Patent Application Publication
(10) Pub. No.: US 2020/0281778 A1
(43) Pub. Date: Sep. 10, 2020

(54) INCONTINENCE DEVICE

(71) Applicant: Health Research, Inc., Buffalo, NY (US)

(72) Inventors: Christopher LEWCZYK, Alden, NY (US); James L. MOHLER, Buffalo, NY (US)

(21) Appl. No.: 16/758,139

(22) PCT Filed: Oct. 26, 2018
(86) PCT No.: PCT/US2018/057871

§ 371 Date: Apr. 22, 2020

Related U.S. Application Data
(60) Provisional application No. 62/577,243, filed on Oct. 26, 2017.

(51) Int. Cl. A61F 13/471; A61F 13/62; A61F 13/78; A61F 13/56

(52) U.S. Cl. CPC A61F 13/471; A61F 13/5611; A61F 13/78; A61F 13/622

(57) ABSTRACT

The present disclosure may be embodied as an incontinence device for a catheterized individual. The device includes an absorbent pad configured to be worn by the individual. The absorbent pad having a slit partially separating the pad into two portions. A fastener may be provided for selectively attaching the two portions to each other. In this way, a catheter may be disposed through the slit of the device and the two portions may be attached to each other using the fastener to surround a circumference of the catheter. The fastener may be reusable. In some embodiments, the device includes more than one fastener. The fastener may be one or more of an adhesive, a hook-and-loop fastener, a snap fastener, or combinations thereof.

### CROSS-REFERENCE TO RELATED APPLICATIONS

This application claims priority to U.S. Provisional Application No. 62/577,243, filed on Oct. 26, 2017, now pending, the disclosure of which is incorporated herein by reference.

### FIELD OF THE DISCLOSURE

The present disclosure relates to absorbent pads used by catheterized individuals.

### BACKGROUND OF THE DISCLOSURE

Urinary catheters are often required after surgical procedures to facilitate discharge of urine. For example, after a prostatectomy, patients are generally required to have a urinary (Foley) catheter for several days or longer. Such catheters are prone to leakage around the outer surface of the catheter, i.e., at the meatus-catheter junction. It is not uncommon for a patient's bladder to spasm due to, for example, the presence of the catheter. When a near-empty bladder spasms, the bladder wall may occlude the catheter openings preventing urine from being discharged normally through the lumen of the catheter. However, the same spasm creates pressure which forces urine to be discharged around the outside of the catheter. This can create an uncomfortable and embarrassing problem for the patient.

To accommodate such leakage, catheterized individuals often use incontinence pads to absorb the urine. However, the use of such pads is not entirely effective because leaked urine often follows along the surface of the catheter and around the edge of the incontinence pad. Accordingly, there continues to be a need for an absorbent pad which is better able to prevent urine from leaking beyond the pad, causing discomfort and embarrassment for a catheterized individual.

### BRIEF SUMMARY OF THE DISCLOSURE

The present disclosure may be embodied as an incontinence device for a catheterized individual. The device includes an absorbent pad configured to be worn by the individual. The absorbent pad having a slit partially separating the pad into two portions. A fastener may be provided for selectively attaching the two portions to each other. In this way, a catheter may be disposed through the slit of the device and the two portions may be attached to each other using the fastener to surround a circumference of the catheter. The fastener may be reusable. In some embodiments, the device includes more than one fastener. The fastener may be one or more of an adhesive, a hook-and-loop fastener, a snap fastener, or combinations thereof.

The pad may have a primary longitudinal axis, and the slit may be arranged at any angle with respect to such longitudinal axis. For example, the slit may be disposed at 0°, 30°, 45°, 60°, 90°, 180°, 270°, or any value therebetween.

In some embodiments, the device is configured to be folded when in use. In such embodiments, the device may include one or more closures configured to selectively secure the two portions of the pad adjacent to each other in a folded configuration of the pad. The one or more closures comprise an adhesive, a hook-and-loop fastener, or a snap fastener, or combinations thereof.

### DESCRIPTION OF THE DRAWINGS

For a fuller understanding of the nature and objects of the disclosure, reference should be made to the following detailed description taken in conjunction with the accompanying drawings, in which:

FIG. 1 is a perspective view of a male incontinence device according to an embodiment of the present disclosure;

FIG. 2 is a perspective view of a device according to another embodiment of the present disclosure and showing a catheter line disposed through a slit;

FIG. 3A is a top view of device of another embodiment; and

FIG. 3B is a top view of the device of FIG. 3A as it would be configured in use.

### DETAILED DESCRIPTION OF THE DISCLOSURE

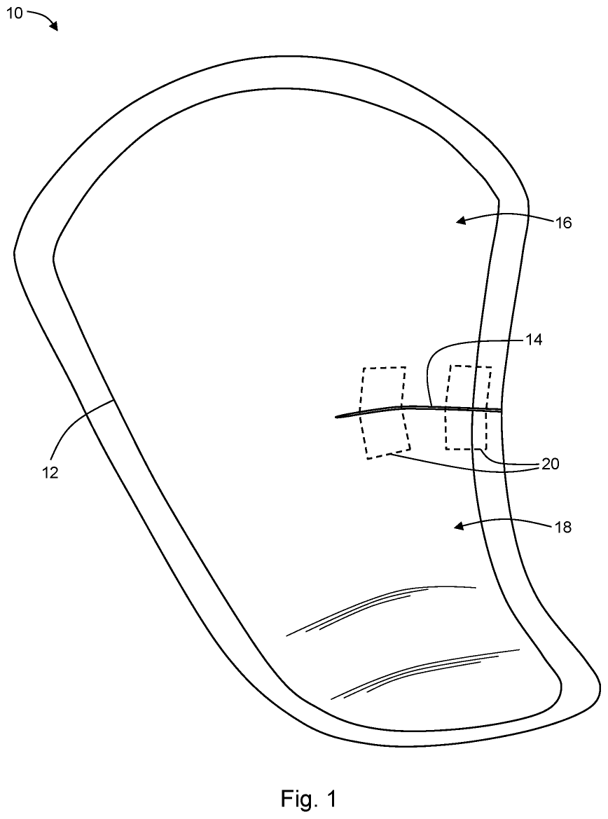

With reference to FIG. 1, the present disclosure may be embodied as a male incontinence device 10 for use by a catheterized individual. The device 10 of the present disclosure comprises an absorbent pad 12 for absorbing urine. The absorbent pad 12 is configured to be worn by the individual. FIG. 1 shows an exemplary configuration intended to be worn within the individual's undergarment. In this way, some embodiments of the device 10 may be held in place by force of the undergarment. Other exemplary absorbent pads may be configured to be worn without the need for securement using an undergarment. For example, the absorbent pad might be configured like underwear and held up by, for example, a waistband. Other configurations will be apparent in light of the present disclosure.

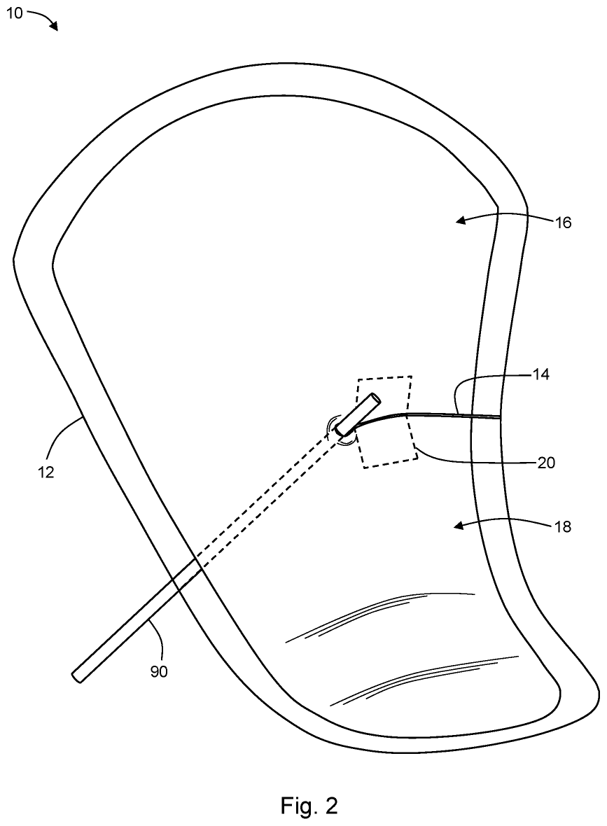

The absorbent pad 12, includes a slit 14 which partially separates the pad 12 into two portions—a first portion 16 and a second portion 18. The slit 14 is configured such that a catheter 90 can be disposed through the slit 14—i.e., such that a circumference of the catheter 90 is effectively surrounded by the pad 12 at a location along the length of the catheter 90 (see, for example, FIG. 2). In embodiments where the absorbent pad has an elongated shape—i.e., along a longitude—the slit 14 may be a transverse slit. In some embodiments, the slit may be arranged at an angle, such as a non-orthogonal angle, with respect to a primary longitudinal axis of the pad. The angle may be any value selected for a particular pad design, intended use, etc. For example, the angle may range from parallel to the longitudinal axis (e.g., 0° or 180° with respect to the axis), to being perpendicular to the longitudinal axis (e.g., 90° or 270°), or at any other angle from 0° to 360°. Although depicted as a generally linear slit, the slit need not be linear. For example, the slit may be curvilinear. In some embodiments, the slit may have a combination of one or more curved and/or linear portions. Such configurations may provide, for example, improved retention of a catheter within the slit, improved performance in blocking urine from migrating along an outer surface of the catheter, etc.

The incontinence device 10 further includes a fastener 20 for selectively attaching the two portions 16, 18 of the pad 12 to each other. In this manner, when a catheter is disposed in the slit 14, the fastener 20 is used to secure the pad 12 around the catheter to prevent urine from traveling past the pad 12. More than one fastener 20 may be used. For example, FIG. 1 shows an embodiment having two fasteners 20. The fastener 20 may be permanent or temporary (i.e., reusable) such that the fastener 20 can be removed and reattached more than one time. The fastener 20 may be of any type suitable for joining the two portions of the pad 12. For example, the fastener may be an adhesive, an adhesive tape, a hook-and-look fastener, a snap fastener, or any other type of fastener or combinations of fastener types.

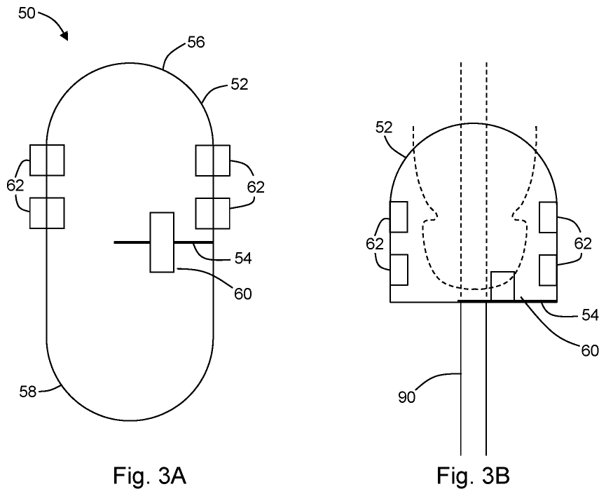

In some embodiments, the incontinence device is intended to be folded on itself and so as to be affixed around the penis of a catheterized male. For example, FIGS. 3A and 3B show an exemplary device 50 having absorbent pad 52 and slit 54, partially dividing the absorbent pad 52 into two portions—first portion 56 and second portion 58. A fastener 60 is provided as described above. The device 50 of this embodiment also includes one or more closures 62 which are configured to affix the first portion 56 adjacent to the second portion 58 when the absorbent pad 52 is folded. The device 50 of the depicted embodiment shows where the absorbent pad 52 is folded at a location that is substantially along the slit 54. However, other similar pads may be folded at locations other than the slit. The absorbent pad 52 is configured to be folded such that the glans of the penis of the individual is between the first portion 56 and the second portion 58. Once the absorbent pad 52 is folded, the closure(s) 62 may be used to affix the first portion 56 to the second portion 58 in much the same way as described above with respect to the fastener. In this way, the device 50 will remain attached to a location on the penis advantageous to prevent leakage of urine beyond the device. The one or more closures 62 may utilize adhesive, hook-and-loop, snap fasteners, etc. FIGS. 3A and 3B show an exemplary embodiment using four closures 62, but any number of closures may be used as will be apparent to one having skill in the art.

Although the present disclosure has been described with respect to one or more particular embodiments, it will be understood that other embodiments of the present disclosure may be made without departing from the spirit and scope of the present disclosure. Hence, the present disclosure is deemed limited only by the appended claims and the reasonable interpretation thereof.

What is claimed is:

1. An incontinence device for a catheterized individual, comprising:
an absorbent pad configured to be worn by the individual, the absorbent pad having a slit partially separating the pad into two portions; and
a fastener for selectively attaching the two portions to each other.

2. The device of claim 1, wherein the fastener is reusable.

3. The device of claim 1, wherein the fastener is one or more of an adhesive, a hook-and-loop fastener, a snap fastener, or combinations thereof.

4. The device of claim 1, wherein the pad has a primary longitudinal axis, and the slit is arranged at an angle of between 0° and 45°, inclusive, or any integer value therebetween, with respect to the longitudinal axis.

5. The device of claim 1, wherein the pad has a primary longitudinal axis, and the slit is arranged at an angle of between 45° and 90°, inclusive, or any integer value therebetween, with respect to the longitudinal axis.

6. The device of claim 1, wherein the pad has a primary longitudinal axis, and the slit is arranged at an angle of between 90° and 180°, inclusive, or any integer value therebetween, with respect to the longitudinal axis.

7. The device of claim 1, wherein the pad is configured to be folded when in use.

8. The device of claim 7, further comprising one or more closures configured to selectively secure the two portions of the pad adjacent to each other in a folded configuration of the pad.

9. The device of claim 7, wherein the one or more closures comprise an adhesive, a hook-and-loop fastener, or a snap fastener.
